(19) United States
(12) Patent Application Publication
(10) Pub. No.: US 2005/0069926 A1
(43) Pub. Date: Mar. 31, 2005

(54) Helicase-amplified reverse transcription

(75) Inventors: Kyle B. Cole, Stanford, CA (US); Vivi Truong, Mountain View, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 10/910,158

(22) Filed: Aug. 2, 2004

Related U.S. Application Data
(60) Provisional application No. 60/491,797, filed on Aug. 1, 2003.

(51) Int. Cl. C12Q 1/68; C12P 19/34

(52) U.S. Cl. 435/6; 435/91.2

(57) ABSTRACT

Methods are provided for enhancing generation of cDNA strands from mRNA. These methods are particularly useful for generating sufficient quantities of target for microarray hybridization.

### RELATED APPLICATIONS

The present application claims priority to U.S. Provisional Application 60/491,797 filed Aug. 1, 2003, the entire disclosure of which is incorporated herein by reference in its entirety.

### FIELD OF THE INVENTION

The invention is related to methods of generating multiple copies of cDNA from mRNA.

### BACKGROUND OF THE INVENTION

There are a number of ways to synthesize first strand cDNA from mRNA (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)). For example, the first strand cDNA can be synthesized by a reverse transcriptase with a primer. Reverse transcriptases are readily available from many sources.

After first strand synthesis of the cDNA is complete, a mRNA-cDNA complex results. From this complex, a second strand of DNA may be fabricated.

### SUMMARY OF THE INVENTION

Methods are provided for enhancing generation of cDNA strands from mRNA. These methods are particularly useful for generating sufficient quantities of target for microarray hybridization. According to the methods, mRNA is provided followed by hybridizing the mRNA with a primer. According to the methods provided, the primer may be either an oligo dT primer or random primers, for example random hexamers. After priming, the synthesis of a cDNA is directed using reverse transcriptase to provide a cDNA-mRNA complex. The complex is thereafter denatured using a helicase to provide a mRNA which can then be used in another round of cDNA synthesis. According to the provided methods, the steps of cDNA synthesis followed by helicase denaturation can be performed a multitude of times to provide a high quantity of cDNA.

### BRIEF DESCRIPTION OF THE DRAWINGS

FIG. 1 depicts helicase-based amplification of reverse transcription.

### DESCRIPTION OF THE PREFERRED EMBODIMENT

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer (anyone have the cite), Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, and 6,136,269, in PCT Applications Nos. PCT/US99/00730 (International Publication Number WO 99/36760) and PCT/US 01/04285, and in U.S. patent applications Ser. Nos. 09/501,099 and 09/122,216 which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

The present invention also contemplates many uses for polymers attached to solid substrates. These uses include gene expression monitoring, profiling, library screening, genotyping, and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefor are shown in U.S. Ser. No. 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. For example, see the patents in the gene expression, profiling, genotyping and other use patents above, as well as U.S. Ser. No. 09/854,317, Wu and Wallace, Genomics 4, 560 (1989), Landegren et al., Science 241, 1077 (1988), Burg, U.S. Pat. Nos. 5,437,990, 5,215,899, 5,466,586, 4,357,421, Gubler et al., 1985, Biochemica et Biophysica Acta, Displacement Synthesis of Globin Complementary DNA: Evidence for Sequence Amplification, transcription amplification, Kwoh et al., Proc. Natl. Acad. Sci. USA 86, 1173 (1989), Guatelli et al., Proc. Nat. Acad. Sci. USA, 87, 1874 (1990), WO 88/10315, WO 90/06995, and U.S. Pat. No. 6,361,947.

The present invention also contemplates detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625 and in PCT Application PCT/US99/06097 (published as WO99/47964), each of which also is hereby incorporated by reference in its entirety for all purposes.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over the internet. See U.S. provisional application 60/349,546.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na⁺], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490 and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Helicase Amplification of Reverse Transcription

Detection of nucleic acid targets from complex samples often utilizes methods of amplifying one or more nucleic acids from the sample prior to analysis. Amplification may be useful for enrichment of one or more specific targets or enrichment of a class of nucleic acid target, for example, enrichment of mRNA. Amplification is also useful for increasing the mass of targets, facilitating detection of targets my downstream analysis methods.

There are many available methods for amplification of nucleic acid sequences, PCR being the most widely used method, see, e.g., U.S. Pat. Nos. 4,683,202, 4,683,195, 4,800,159, 4,965,188 and 5,333,675. In addition to PCR, other amplification methods include the ligase chain reaction (LCR) (e.g., Wu and Wallace, Genomics 4, 560 (1989) and Landegren et al., Science 241, 1077 (1988)), transcription amplification (Kwoh et al., Proc. Natl. Acad. Sci. USA 86, 1173 (1989)), self-sustained sequence replication (Guatelli et al., Proc. Nat. Acad. Sci. USA, 87, 1874 (1990)), rolling circle amplification and nucleic acid based sequence amplification (NABSA), (see, U.S. Pat. Nos. 5,409,818, 5,554,517, and 6,063,603.

RNA may be amplified by first generating cDNA by reverse transcription and then amplification of the cDNA by, for example, PCR or by T7 RNA polymerase as described in U.S. Pat. Nos. 5,545,522 and 6,582,906 and U.S. patent application Ser. Nos. 09/634,352, 10/763,414, 10/877,544, 10/090,320 and 60/514,872. Nucleic acid amplification using strand displacing polymerases has also been shown, see for example, Lasken and Egholm, Trends Biotechnol. 2003 21(12):531-5; Barker et al. Genome Res. 2004 May;14(5):901-7; Dean et al. Proc Natl Acad Sci USA. 2002; 99(8):5261-6; and Paez J G et al. Nucleic Acids Res. 2004; 32(9):e71.

In accordance with one aspect of the present invention, a method for amplifying RNA by generating multiple cDNA strands from mRNA, is provided having the steps of a) providing a mRNA; b) hybridizing the mRNA with a primer; c) directing the syntheses of a cDNA with a reverse transcriptase to provide a cDNA-mRNA complex; d) denaturing the cDNA-mRNA complex with a helicase enzyme activity to provide an uncomplexed mRNA; and e) repeating steps b) to e) to provide a desired quantity of cDNA.

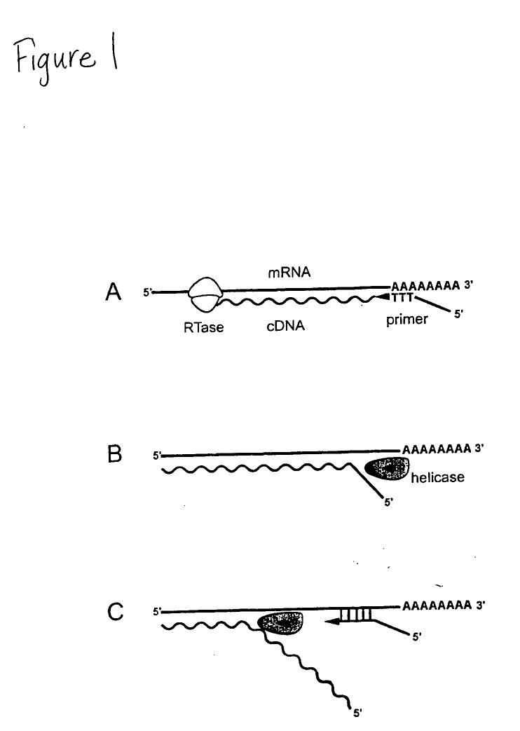

In accordance with one aspect of the present invention it is preferred that the mRNA has a poly A⁺ tail. In accordance with the present invention, an oligo dT primer may be used to prime the synthesis of the cDNA strand when the RNA has a poly A⁺ tail. Such priming is shown in FIG. 1 A.

It is not necessary, however, to use oligo dT priming with respect to the instant invention, in accordance with the present invention, it is also preferred to use random primers. Preferably, the random primers are hexamers. (See FIG. 1, C, showing priming internally). Target specific primers, degenerate primers or partially degenerate primers may also be used.

Reverse transcriptase may be any enzyme that is capable of synthesizing a corresponding cDNA from an RNA template in the presence of the appropriate primers and nucleoside triphosphates. In a preferred embodiment, the reverse transcriptase may be from avian myeloblastosis virus (AMV), Moloney murine leukemia virus (MMuLV) or Rous Sarcoma Virus (RSV), for example, and may be a thermal stable enzyme (e.g., rTth DNA polymerase available from Applied Biosystems, Foster City, Calif.). Commercially available reverse transcriptases include SuperScript™ (Invitrogen, Carlsbad, Calif.).

In accordance with the presently disclosed methods, a variety of helicases may be used. In accordance with one aspect of the methods, a 5′ to 3′ directed DNA helicase is preferred. 3′ to 5′ directed RNA helicases are also preferred in accordance with the present methods.

As shown in FIG. 1C, use of a helicase to unwind the cDNA-mRNA complex allows primers to bind to the mRNA and a second round of reverse transcription to begin. This cycle may be repeated to provide multiple copies of cDNA.

A number of possible helicases have been identified for possible use with respect to the instant invention including, by way of example, T4 DNA Helicase, NS3 RNA helicase of HCV, NSR and SEN 1. To function optimally in the context of the instant invention, a helicase should have sufficient denaturation activity in the buffer conditions that are compatible with reverse transcription. In the most preferred embodiments of the invention, the helicase will retain substantial levels of denaturation activity in buffer conditions used for reverse transcription. However, helicases may still preferably be used in accordance with the present invention where sufficient helicase activity is left to allow reasonable quantities of second and third round generation of cDNA. The cDNA may be further amplified by any suitable method.

In a preferred embodiment the multiple copies of cDNA generated by the disclosed methods are analyzed by hybridization to an array of probes. Preferably the amplified cDNA is fragmented and labeled prior to hybridization. Labeling may be with a fluorescent or chemiluminescent label, for example.

The nucleic acids generated by the methods may be analyzed by hybridization to nucleic acid arrays. Those of skill in the art will appreciate that an enormous number of array designs are suitable for the practice of this invention. High density arrays may be used for a variety of applications, including, for example, gene expression analysis, genotyping and variant detection.

For gene expression analysis, the high density array will typically include thousands or hundreds of thousands of probes that specifically hybridize to the nucleic acid(s) whose expression is to be detected or to its complement. Array based methods for monitoring gene expression are disclosed and discussed in detail in U.S. Pat. Nos. 5,800,992, 5,871,928, 5,925,525, 6,040,138 and PCT Application WO92/10588 (published on Jun. 25, 1992). Generally these methods of monitoring gene expression involve (1) providing a pool of target nucleic acids comprising RNA transcript(s) of one or more target gene(s), or nucleic acids derived from the RNA transcript(s); (2) hybridizing the nucleic acid sample to a high density array of probes and (3) detecting the hybridized nucleic acids and calculating a relative expression (transcription, RNA processing or degradation) level. Suitable arrays are available, for example, from Affymetrix, Inc. (Santa Clara, Calif.).

All patents, patent applications and publications cited in this application are hereby incorporated by reference in their entirety for all purposes to the same extent as if each individual patent, patent application or publication were so individually denoted.

### EXAMPLES

Double stranded DNA was used as a template. Each reaction contained DNA template, a helicase primer (20 bp binds to template leaving a 20 nt tail for the helicase to initiate denaturation), a strand-displacing DNA polymerase (BstI or Klenow exo-), T4 DNA helicase, and random hexamers. As shown Table 1, control reactions that do not have helicase or polymerase do not show amplification.

TABLE 1

| Reaction Conditions | Yield in μg DNA |
|---|---|
| No dNTPs and dTTP/3 hrs | 0.27 |
| No dNTPs and dTIP/8 hrs | 0.097 |
| No DNA/3 hrs | 1.4 |
| No DNA/8 hrs | 1.24 |
| No Helicase/3 hrs | 0.194 |
| No Helicase/8 hrs | 0.404 |
| No Polymerase/3 hrs | 0.09 |
| No Polymerase/8 hrs | 0.325 |
| Random Hex (300 ng)/Bst/3 hrs | 1.63 |
| Random Hex (300 ng)/Bst/8 hrs | 1.89 |
| Random Hex (300 ng)/Klenow exo-/3 hrs | 1.21 |
| Random Hex (300 ng)/Klenow exo-/8 hrs | 2.27 |
| Standard/BST Pol/3 hrs | 1.29 |
| Standard/BST Pol/8 hrs | 1.86 |
| Standard/Klenow exo-/3 hrs | 1.09 |
| Standard/Klenow exo-/8 hrs | 2.27 |

1. A method for generating amplified cDNA strands from mRNA, said method comprising:
a) providing a mRNA;
b) hybridizing said mRNA with a primer;
c) directing the syntheses of a cDNA with a reverse transcriptase to provide a cDNA-mRNA complex;
d) denaturing the cDNA-mRNA complex with a helicase enzyme to provide an uncomplexed mRNA; and
e) repeating steps b) to e) to provide a desired quantity of cDNA.

2. A method for generating amplified cDNA strands according to claim 1, wherein said mRNA has a poly A⁺ tail.

3. A method for generating amplified cDNA strands according to claim 2, wherein said primer is an oligo dT primer.

4. A method for generating amplified cDNA strands according to claim 1, wherein said primer comprises random hexamer primers.

5. A method for generating amplified cDNA strands according to claim 1, wherein said helicase is a 5′ to 3′ directed DNA helicase.

6. A method for generating amplified cDNA strands according to claim 1, wherein said helicase is a 3′ to 5′ directed RNA helicase.

7. A method for generating amplified cDNA strands according to claim 1, wherein steps b) to e) are isothermal.

8. A method for analyzing a nucleic acid sample containing mRNA, the method comprising:
a) contacting the nucleic acid sample with a primer comprising oligo dT;
b) extending the primer with a reverse transcriptase to generate cDNA-mRNA complexes;
c) denaturing the cDNA-mRNA complexes with a helicase enzyme to provide an uncomplexed mRNA;
d) repeating steps a) to d) to provide a desired quantity of amplified cDNA;
e) fragmenting and labeling the amplified cDNA;
f) hybridizing the fragmented, labeled cDNA to an array of probes and detecting a resulting hybridization pattern.

9. The method of claim 8 wherein said helicase is a 5′ to 3′ directed DNA helicase.

10. The method of claim 8 wherein said helicase is a 3′ to 5′ directed RNA helicase.
